(19) United States
(12) Patent Application Publication
(10) Pub. No.: US 2002/0086837 A1
(43) Pub. Date: Jul. 4, 2002

(54) Acne vaccine

(76) Inventors: Jack Gauldie, Hamilton (CA); Todd Braciak, Hamilton (CA)

Correspondence Address: VAN DYKE & ASSOCIATES, P.A., 1630 HILLCREST STREET, ORLANDO, FL, 32803, US

(21) Appl. No.: 09/742,892

(22) Filed: Dec. 21, 2000

(51) Int. Cl. A61K 48/00; A61K 7/48

(52) U.S. Cl. 514/44; 424/401

(57) ABSTRACT

Disclosed herein are vectors and methods for use in the prevention and treatment of acne. Specifically exemplified are adenovirus vectors or plasmid DNA that encode a variety of target antigens from proprionibacterium acnes with or without cytokines, chemokines or co-stimulatory receptors. These vectors in both viral and DNA vector form are prolific molecular adjuvants and can be used in combination with vectors and DNA encoding the lipase, hyaluronidase, phosphatase and other genes on the same operon, to elicit protective immune responses against colonization of the bacterium in the skin follicles.

### BACKGROUND OF THE INVENTION

Acne vulgaris is a multifactorial skin disease which is generally characterized by the presence of a variety of inflamed and noninflamed lesions on the face and upper trunk. Although the disease occurs mainly during adolescence, it should now no longer be solely viewed as a condition of pubescence. A considerable number of patients have persistent cases of acne into their 30's and even into their 50's. While the precise role of bacterial colonization, sebum production, immune factors, genetics and hormonal changes are not yet entirely clarified, the skin bacterium Propionibacterium acnes has been increasingly implicated in the pathogenesis of inflammatory acne (whiteheads).

It has been demonstrated that chemical agents including antibiotics, which reduce the numbers of this organism are therapeutic. In fact, it has been shown in patients failing to respond to erythromycin antibiotic therapy that P. acnes bacterium isolated from lesions of the skin acquired antibiotic resistance. Another agent benzoyl peroxide, the active ingredient in many of the over-the-counter acne products, has also been directly correlated with its effects on P. acnes colonization in the skin. Thus, it appears that in inflammatory acne, interference with colonization of skin follicles by the P. acnes bacterial species can prevent the most common form of inflammatory acne (whiteheads). This process of bacterial colonization of the skin follicle may provide a target for the acne gene therapy and vaccine.

The underlying basis of acne appears to be associated with the presence of larger than normal sebaceous follicles coupled with an increased level of sebum production from enlarged sebaceous glands (i.e. the response of the skin to the presence of the bacteria). In addition, keratinization (the process of skin formation) in the follicle is abnormal and leads to the generation of ‘sticky’ cells which can occlude the follicle. This blockage in combination with excess sebum production can facilitate the colonization of the follicle by Propionibacterium acnes. P. acnes organisms are able to metabolize the readily abundant triglycerides (fats) present in the sebum, and fatty acid by-products from this metabolism can contribute to the inflammatory acne condition in at least two ways. First, the metabolic breakdown products of the fatty acids have been shown to facilitate the colonization and survival of the P. acnes bacterium in the follicle. Secondly, these fatty acid molecules can be converted into chemoattractants (molecules which attract and recruit immune and inflammatory cells of the body) producing inflammation. It is dependent on the degree and intensity of this process that the more severe superficial pustules and papules of acne may form resulting in skin destruction and scarring.

Regarding other possible etiologic factors, it is upon the basis of abnormal sebum production that hormonal changes in androgen (testosterone) production are thought to affect acne. Sebum production from sebaceous glands in the skin follicle is stimulated by androgenic metabolites. Aspects of genetic susceptibility may be manifested by the underlying effects on the genetic control of host immune and inflammatory function.

As described above, the condition of inflammatory acne has been linked to colonization of the skin follicle by the bacterial species Propionibacterium acnes. The inventors have discovered that this bacterium offers a number of targets for immunotherapy. However, since the basis of the therapy is to induce an immune response to bacterial antigens, the selection of the target antigen must be judicious since there is skepticism in the medical community that immunotherapy is a viable therapeutic regimen. For example, previous scientific reports have implicated active but not effective immune responses to the P. acnes bacterium as being contributory to the acne disease process. Karvonen et al., Dermatology 189:344-349 (1994); and Holland et al, Exp. Dermatol.2:12-16 (1993).

Innate immune responses are typified by specialized cells that respond and are activated to generalized features of a pathogen. One of these special cell types, the macrophage, plays an important role in the pathology of acne and the colonization of skin follicles by the P. acnes bacterium. Generally, macrophages are able to respond to bacterial signals without having encountered the pathogen before (innate response) by phagocytosing (engulfing and destroying) the pathogen. In the acne condition, the P. acnes bacterium has evolved mechanisms to avoid this destruction. Thus, macrophages in the skin follicles of an inflamed acne lesion serves as a reservoir for the bacterium, with many of the organisms residing within the body of the cell.

T cells, on the other hand, are an important component of the immune system arsenal, in that these cells are able to kill pathogens which have evolved mechanisms which allow them to be harbored inside cells of the body (cell mediated immunity), like the macrophages harboring the P. acnes bacterium.

It is therefore important to identify the type of immune response and the correct target antigen on the pathogen in order to eliminate acne pathology. The inventors have identified and cloned novel antigens of the P. acnes bacterium which can be used to induce protective immune responses. These antigens have not been implicated in the destructive form of acne and in fact, responses against these antigens have not been detected in patients whose unsuccessful immune response to P. acnes was characterized. We believe the molecules we have identified are essential for the organism's growth and colonization of the skin follicle and the eventual inflammatory acne pathology (whiteheads).

### SUMMARY OF THE INVENTION

Accordingly, it is an object of the invention to develop new approaches and vaccines for gene therapy of diseases. It is particularly an object of the invention to develop new therapies that are useful in treating acne and other pathological conditions. It is specifically an object to explore the use of a series of recombinant adenoviral vectors which contain genes encoding various target antigens and/or immunostimulatory proteins to introduce into target tissues nucleic acids which can be used to treat diseased conditions in the host.

A specific aspect of the subject invention pertains to recombinant adenovirus vectors and/or recombinant viral particles that include, and support the replication and expression of, nucleic acids that encode both targeted pathogen antigens and, optionally, immunostimulatory molecules such as cytokines and co-receptors that enhance immunostimulation and/or chemotactic factors which recruit lymphocytes. These vectors can be administrated whereby expression of the cytokine and co-stimulatory molecule stimulate an immune response against the targeted antigens. Where the adenovirus expresses a chemokine as well, this vector will produce chemotactic factors within the injection site and would recruit T cells from the circulation into the tissue and initiate immunity. T cells stimulated in either scenario will react against the bacterial antigen resulting in destruction of bacteria.

Adenoviruses (Ads) have several properties that make them attractive for gene therapy. They can be grown to high titers. Human Ads can infect a variety of cell types, from a variety of species. In particular, adenovirus type 5 (Ad5) naturally infects human cells and can be used as a vector for the delivery of foreign genes to human tissues. Transduced genes can be expressed in non-dividing cells. A deletion of Ad early region 1 (“E1”) can be combined with one in Ad early region 3 (“E3”) (whose products are unnecessary for growth in culture); together these deletions increase the packaging capacity of the modified Ad to accommodate up to 8 kb of foreign DNA. Several non-defective and replication defective (E1-) have been characterized (see, e.g., Verna and Somia, Nature 389; 239-242 (1997); the E1 vectors are able to replicate only in certain cell lines such as the 293 cell line. El Ads can persist and continue to express in cultured cells and in vivo for extended periods. Since the virus does not integrate efficiently, expression should be transient, a possible advantage for certain gene therapy applications, particularly immunotherapy of bacterial infection.

In a preferred embodiment, the subject invention is directed to a genetic vaccine utilizing recombinant vectors, such as the adenovirus vector system or naked DNA. Specifically exemplified are vectors comprising nucleotide sequences encoding P. acnes proteins and fragments thereof. According to one aspect, the subject invention pertains to a vector comprising a lipase gene, a hyluronidase gene and/or phosphatase gene from P. acnes, as well as other yet uncharacterized genes, to target bacterial antigens and induce T cell responses (cell mediated immunity) that kill macrophages which contain bacteria. Simultaneously, B cell responses are induced (antibodies) which can neutralize the bacterium and interfere with colonization of the skin follicle. We have documented the efficiency of the adenovirus vector system for its capability of inducing such a response.

A further aspect of the subject invention pertains to production of a vaccine and method of administration involving, for example, intramuscular injection or direct application (e.g., via patch) to the skin and therefore, minimize the concern of immunotoxicity. These modes of administration have been shown to produce transgene product even in the presence of antibodies which may already be present due to prior exposure of the individual to the wild type virus in nature.

The subject vaccination system provides several advantages over all other gene therapy approaches developed to date. Those skilled in the art will appreciate that commercially available gene therapy vectors (e.g., recombinant retrovirus, adenovirus associated virus; AAV, and DNA itself) can be used in accord with the principles of the subject invention. However, the adenovirus vector is a preferred vector, as it has several advantages. First, an ad vector is highly efficient in transferring genetic material (DNA) to the target cell. The virus genome (its genetic material which encodes the virus proteins) contains DNA and must enter the nucleus of infected cells in order to replicate. Thus, the structural molecules of the virus have evolved to facilitate the most efficient delivery for the viral DNA to the nucleus. Since in the recombinant virus we have cloned the gene we want encoded and expressed into the viral genome, this process of transfer can be matched by no other vector. Second, ad vectors have the ability to carry large segments of DNA (genetic information) up to 30,000 base pairs can be carried by a recombinant vector. Our typical target bacterial antigen is around 1000 base pairs which is similar in size to that of a cytokine molecule. Thus, a single vaccine could accommodate as many as 30 different bacterial target antigens or immune modifying cytokines, or any combination of both. Third, ad vectors have the ability to infect non-dividing cells. Other gene therapy vectors can only target dividing cells and would render them useless for targeting to muscle tissue as muscle cells are not an actively dividing cell type. Fourth, ad vector gene expression is transient in the target cell due to the lack of integration of the viral DNA into the host cell DNA. This is a highly desirable attribute of adenovirus vectors since longterm expression of immunomodulating molecules can be harmful. Other vectors are integrated into chromosomes and could cause insertional inactivation or mutation of genes in such treated individuals.

As discussed, a preferred agent for acne gene therapy is a recombinant adenovirus expressing targeted antigens from the P. acnes bacterium. In an alternative embodiment, the agent is naked DNA. Use of naked DNA itself encoding various genes has been successfully used to induce immune responses. The use of DNA as a molecular medicine is receiving considerable attention in the art. In addition to being used as the primary vector for vaccination, DNA can be used as a suitable booster to recombinant adenovirus vaccination.

Futher, the efficacy of the subject vaccination system can be bolstered by co-expression or co-administration of cytokines serving as powerful adjuvants in combination with the bacterial antigen targeted vector. Studies have shown that a class of soluble protein hormones called cytokines possess anti-tumor and anti-metastatic activity due to their ability to activate immune cells, such as T cells, which can recognize foreign elements unique to the tumor, known as antigens. T cells are capable of selectively attacking tumor tissue, leaving the normal tissue relatively unharmed. They can circulate throughout the body where they will identify tumor cells which have disseminated from the primary tumor site and destroy them. These same responses can be targeted toward attack of pathogenic bacteria. Cytokines are normally produced during the effector phases of natural and specific immunity. They mediate and regulate immune and inflammatory processes by stimulating cells that participate in an immune response to migrate and accumulate in inflamed tissues, by activating cellular functions that mediate the immune response, and by causing immune system cells to liberate signalling and effector substances. However, cytokines alone may not be sufficient for full activation of T cell responses which underly the specific component of adaptive immunity. Antigen presenting cells, those cells which are required to initiate T cell responses, express surface receptors, referred to as co-stimulatory molecules, which synergize with cytokines in the activation of T cells. For example, the cytokines interleukin-2 (IL-2) and interleukin-12 (IL-12) can promote anti-tumor activity through their ability to stimulate the cytolytic activity of T-cells, LAK cells (lymphokine activated killer cells), and TILS (tumor infiltrating lymphocytes). IL-2 is produced primarily by activated T-lymphocytes and by natural killer cells (NK) or LAK cells and acts in an autocrine and paracrine fashion to augment an immune response. It exerts regulatory effects on almost all cell types involved in immune responses; it stimulates the proliferation and differentiation of B-cells, T-cells, NK cells, LAK cells as well as the activation of monocytes and macro phages. It can also stimulate the production of other cytokines such IFN-g and TNF-a (Anderson, T. D., 1992, in: Cytokines in Health and Disease (eds. S. L. Kunkel and D. G. Remick); Marcel Dekker Inc., New York, pp.27-60). IL-12 on the other hand is secreted by professional antigen presenting cells (APCs) and serves to direct the development of immature T cells towards a T1-type cytokine profile which is characterized by the secretion of IFN-g and IL-2. IL-12 is a very potent inducer of IFN-g which accounts in part for its anti-tumor properties (Brunda, M. J. et al., 1995, J. Immunother. 17: 71). We have used the adenovirus expressing IL 12 to protect mice against L. major parasitic infection. Raja Gabaglia et al., J Immunol 162:753-760 (1999). This IL-12 treatment prevented the intracellular infection of macrophages by parasites and is a proof in principle that this vector will be a suitable adjuvant to prevent P. acnes colonization.

The mature form of IL-2 is a 133 amino acid secreted protein which ranges in molecular weight from 14 to 18 kD due to differential glycosylation. Human IL-2 is biologically active in a wide variety of species, and thus testing of its anti-tumor properties in various animal models is possible. IL-12 is a more complex protein consisting of two separate subunits of 35 and 40 kD in size. Both subunits are heavily glycosylated and they are linked in a 1:1 ratio by disulfide bonds. The murine cytokine is active in many species and thus it too can be used in a variety of animal models.

In order to be an effective anti-cancer treatment, high levels of cytokine are required at the site of the tumor. However, when delivered through the systemic circulation, high levels of cytokines such as IL-2 or IL-12 can result in immunotoxicities. To avoid elevated concentrations of circulating cytokines, we have developed adenovirus vectors which express either IL-2 or IL-12 and we have shown them to be highly useful in the treatment of murine breast carcinoma. (Addison, C.L.et al, 1995, Proc. Natl. Acad. Sci. USA 92: 8522; Bramson et al., 1996, Hum. Gene Ther. 7:1995). By injecting the adenovirus directly into the tumor nodule, we are able to induce very high levels of cytokine expression with very little secretion into the serum (Bramson et al., 1996, Hum. Gene Ther. 7:1995). However, these treatments only lead to cures in 30-40% of the animals following a single inoculation. One way to improve the outcome of the therapy is to increase the immunogenicity of the tumor. In that way the tumor becomes a “better target” for the immune cells which are activated by the adenovirally delivered cytokine.

In the course of antigen presentation to a T cell, a number of sequential contacts need to be made between the T cell and the antigen presenting cell (APC) in order to ensure full activation and function of the T cell. These interactions occur between the major histocompatibility molecule-antigen complexes on the APC and the T cell receptor on the T cell. However, simply ligating the T cell receptor is insufficient for proper activation. The T cell must also ligate other molecules on the APC known as co-stimulatory molecules, including the B7-family of proteins and CD40. In the absence of co-stimulation, the T cell enters a state of non-responsiveness known as anergy. It has been clearly demonstrated in multiple models that the immunogenicity of tumors can be enhanced by the expression ofB7-1. Similarly another B7 family member, B7-2, can also improve tumor rejection. Thus, in the subject system, the addition of a co-stimulatory molecule such as B7-1 should improve anti-bacteria responses.

These and other advantageous aspects of the subject invention are discussed in further detail below.

### BRIEF DESCRIPTION OF THE DRAWINGS

FIG. 1 shows a schematic of the production of a viral vector (Ad5E1PBAL) according to the teachings herein, which is capable of preventing and/or treating Acne Vulgaris.

FIG. 2 represents a graph that demonstrates pre-immunization with the subject vaccine provides protection against P. acnes.

### DETAILED DESCRIPTION OF THE PREFERRED EMBODIMENTS

### Definitions

All technical and scientific terms used herein, unless otherwise defined, are generally intended to have the same meaning as commonly understood by one of ordinary skill in the art. A number of the terms used herein are not intended to be limiting, even though common usage might suggest otherwise. For example, the term “expression of” or “expressing” a foreign nucleic acid, gene or cDNA is used hereinafter to encompass the replication of a nucleic acid, the transcription of DNA and/or the translation of RNA into protein, in cells or in cell-free systems such as wheat germ or rabbit reticulocyte lysates; and “nucleic acid” is used interchangeably with gene, cDNA, RNA, or other oligonucleotides that encode gene products. The term “foreign” indicates that the nucleic acid is not found in nature identically associated with the same vector or host cell, but rather that the precise association between the said nucleic acid and the vector or host cell is created by genetic engineering. The term “recombinant” and “recombination” generally refer to rearrangements of genetic material that are contemplated by the inventors, and that are the result of experimental manipulation.

By “capable of facilitating an immune response”, it is meant that a molecule(s) stimulates a cell(s) that participate in an immune response to migrate into and accumulate at tissues in which the molecule is present, and/or that said molecule(s) is (are) capable of stimulating cells of the immune system to engage in activities such as phagocytosis and cytolysis that are part of an immune response, and/or that said molecule(s) cause(s) immune system cells to liberate signalling and effector substances such as, for example, cytokines, antibodies and histamines.

“Vector” as used herein denotes a genetically engineered nucleic acid construct capable of being modified by genetic recombinant techniques to incorporate any desired foreign nucleic acid sequence, which may be used as a means to introduce said sequence in a host cell, replicate it, clone it, and/or express said nucleic acid sequence, wherein said vector comprises all the necessary sequence information to enable the vector to be replicated in host cells, and/or to enable the nucleic acid sequence to be expressed, and/or to enable recombination to take place, and/or to enable the vector to be packaged in viral particles. This recitation of the properties of a vector is not meant to be exhaustive. Those skilled in the art will understand that the use of the term “vector”, and its plural “vectors”, can be used interchangeably, and where appropriate refer to one or more vectors as described herein.

Vectors, optionally containing a foreign nucleic acid, may be “introduced” into a host cell, tissue or organism in accordance with known techniques such as transformation, transfection using calcium-phosophate precipitated DNA, electroporation, particle bombardment, transfection with a recombinant virus or phagemid, infection with an infective viral particle, injection into tissues or microinjection of the DNA into cells or the like. Both prokaryotic and eukaryotic hosts may be employed, which may include bacteria, yeast, plants and animals, including human cells.

Once a given structural gene, cDNA or open reading frame has been introduced into the appropriate host, the host may be grown to express said structural gene, cDNA or open reading frame. Where the exogenous nucleic acid is to be expressed in a host which does not recognize the nucleic acid's naturally occurring transcriptional and translational regulatory regions, a variety of transcriptional regulatory regions may be inserted upstream or downstream from the coding region, some of which are externally inducible. Illustrative transcriptional regulatory regions or promoters for use in bacteria include the p-gal promoter, lambda left and right promoters, trp and lac promoters, trp-lac fusion promoter, and also the bacteriophage lambda Q operator and the C1857 temperature-sensitive repressor, for example, to provide for temperature sensitive expression of a structural gene. Regulation of the promoter is achieved through interaction between the repressor and the operator. For use in yeast, illustrative transcriptional regulatory regions or promoters include glycolytic enzyme promoters, such as ADH-I and -II promoters, OPK promoter, and POI promoter, TRP promoter, etc.; for use in mammalian cells, transcriptional control elements include SV 40 early and late promoters, adenovirus major late promoter, etc. Other regulatory sequences useful in eukaryotic cells can include, for example, the cytomegalovirus enhancer sequence, which can be fused to a promoter sequence such as the SV40 promoter to form a chimeric promoter, or can be inserted elsewhere in the expression vehicle, preferably in close proximity to the promoter sequence. Where the promoter is inducible, permissive conditions may be employed (for example, temperature change, exhaustion, or excess of a metabolic product or nutrient, or the like).

When desired, expression of structural genes can be amplified by, for example, ligating in tandem a nucleic acid for a dominant amplifiable genetic marker 5′ or 3′ to the structural gene and growing the host cells under selective conditions. An example of an amplifiable nucleic acid is the gene for dihydrofolate reductase, expression of which may be increased in cells rendered resistant to methotrexate, a folate antagonist.

The expression vehicles used or provided herein may be included within a replication system for episomal maintenance in an appropriate cellular host, they may be provided without a replication system, or they may become integrated into the host genome.

While a wide variety of host cells are contemplated, certain embodiments require that the host cell express sequences that are missing from or inactivated in the vector. While the human 293 cell lines is the preferred host cell, the invention also contemplates other cell lines capable of complementing the vector having an El deletion. “Complementing” or “complemented by” denotes that the host cell line encodes and/or expresses functions that are necessary for generating viable viral particles that are missing from or have been inactivated in the vector.

It is important to recognize that the present invention is not limited to the use of such cells specifically exemplified herein. Cells from different species (human, mouse, etc.) or different tissues (breast epithelium, colon, neuronal tissue, lymphocytes, etc . . . ) may also be used.

The term “pathogen” as used herein refers to viruses, bacteria, fungi, protozoa, parasites, or other microbes, organisms and agents that infect cell(s) and tissues thereby causing disease or other adverse symptoms. As particularly used herein, the term “pathogen” preferably refers to agents that have the capability to infect, or avoid destruction by, macrophages. Examples of such agents include, but are not limited to, P. acnes, L. monocytogenes, S. typhimurium, N. gonorrhoea, M avium, M. tuberculosis, M leprae, B. abortus, and C. albicans; and L. major,

“Modification” of a nucleic acid indicates all molecular alterations of a nucleic acid sequence that change its capacity to perform a stated function, specifically including deletions, insertions, chemical modifications and the like. Insertions and deletions may be made in a number of ways known to those skilled in the art, including enzymatically cutting the full length sequence followed by modifications and ligation of defined fragments, or by site-directed mutagenesis, especially by loop-out mutagenesis of the kind described by Kramer et al,. 1984, Nucl. Acid Res. 12: 9441-9456.

“Fragment” or “subfragment” refers to an isolated nuclecic acid derived from a reference sequence by excising or deleting one or more nucleotides at any position of the reference sequence using known recombinant techniques, or by inserting a predetermined sequence of nucleotides at any predetermined position within the reference sequence using known recombinant techniques.

It is not intended that the invention be limited to the use of nucleic acid sequences from any particular species or genus, but that this invention can be carried out using nucleic acids from a variety of sources. It is contemplated that any nucleic acid from any source may be inserted into the vector, with or without control elements.

“Gene therapy” comprises the correction of genetic defects as well as the delivery and expression of selected nucleic acids in a short term treatment of a disease or pathological condition. Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, etc., such that a different but known way is used to achieve the same goals as those to which the use of a suggested method, material or composition is directed.

The present invention is not limited to the use of all of the described discoveries or embodiments explicitly described herein. Although combining them may indeed be preferred, it is not necessary to the invention that all aspects be used simultaneously.

The isolated nucleic acids of this invention can be used to generate modified polypeptides, each having at least one characteristic of the native polypeptide. These include subfragments, deletion mutants, processing mutants, or substitution mutants, polypeptides having the same secondary structure as the binding region of the native polypeptide, and combinations thereof. Such modified polypeptides may carry the functionality of the “wild type” peptide, or may have a modified or externally regulatable functionality. Such modified polypeptides may have considerable utility in the present invention, as would be appreciated by those skilled in the art.

“Wild type”, mutant and analogous polypeptides and compositions thereof may be used for making antibodies, which may find use in analyzing results of the assays described as part of this invention. The antibodies may be prepared in conventional ways either by using the subject polypeptide as an immunogen and injecting the polypeptide into a mammalian host, e.g., mouse, cow, goat, sheep, rabbit, etc., particularly with an adjuvant, e.g. complete Freund's adjuvant, aluminum hydroxide gel, or the like. The host may then be bled and the blood employed for isolation of polyclonal antibodies, or the peripheral blood lymphocytes (B-cells) may be fused with an appropriate myeloma cell to produce an immortalized cell line that secretes monoclonal antibodies specific for the subject compounds.

“Cosmetic agent” as used herein pertains to its commonly understood meaning and relates to known materials used in cosmetics designed for application to the skin for purposes of covering, hiding, or lessening the appearance of deficits, blemishes, sores or other defects present on the user's skin.

### Modes of Administration

Those skilled in the art will appreciate that the vectors, polynucleotides and/or polypeptides of the subject invention can be administered by a number of widely recognized and known methods of administration. The subject vectors can be administered prophylactically, or to patients having a disease or condition treatable by supplying and expressing a particular therapeutic nucleic acid sequence or sequences. Routes of administration may include intramuscular, intravenous, aerosol, oral (tablet or pill form), topical, transcutaneous, systemic, ocular, as a suppository, intraperitoneal and/or intrathecal. The specific delivery route of a given vector will naturally depend on the type of vector used and its intended purpose.

To enhance cellular uptake, the subject vectors may be modified in ways which reduce their charge but will maintain the expression of specific functional groups in the final translation product. This results in a molecule which is able to diffuse across the cell membrane, thus removing the permeability barrier.

Chemical modifications of the phosphate backbone may be performed that reduce the negative charge allowing free diffusion across the membrane. This principle has been successfully demonstrated for antisense DNA technology which shows that this is a feasible approach. In the body, maintenance of an external concentration will typically be necessary to drive the diffusion of the modified nucleic acid sequence encoding the subject vectors into the cells of the tissue. Intravenous administration with a drug carrier designed to increase the circulation half-life of the subject vectors can also be used. In addition to controlling the rate of uptake, the carrier can protect the subject vectors from degradative processes.

Drug delivery vehicles are effective for both systemic and topical administration of nucleic acids and polypeptides. They can be designed to serve as a slow release reservoir, or to deliver their contents directly to the target cell. An advantage of using direct delivery drug vehicles is that multiple molecules are delivered per uptake. Such vehicles have been shown to increase the circulation half-life of drugs which would otherwise be rapidly cleared from the blood stream. Some examples of such specialized drug delivery vehicles which fall into this category are liposomes, hydrogels, cyclodextrins, biodegradable nanocapsules, and bioadhesive micro spheres.

In a preferred embodiment, the subject vectors are provided on a patch that can be adhered to the skin of the patient. This novel approach allows for an easy, noninvasive method of delivering the subject vectors to target cells and tissues. This patch delivery method uses the innate properties of the skin to provide prophylactic and therapeutic access to the skin's immune system. Naturally, this patch method of delivery will have certain appeal to recipients suffering from a skin disorder such as acne vulgaris. Preferably, the area intended to receive the patch can be pretreated to increase and enhance the permeability of the skin. Examples of materials that can be used to pretreat the skin include water, alcohol, hydrogels and other known permeation enchancers. Additionally, or alternatively, the patch is separately but concurrently administered with, a permeation enhancer. The patch can be adhered to the skin with known adhesives commonly used in the art.

Alternatively, the subject vectors, polynucleotides and/or polypeptides are administered using liposomal technology. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acid remains biologically active. For example, a liposome delivery vehicle originally designed as a research tool, Lipofectin, has been shown to deliver intact mRNA molecules to cells yielding production of the corresponding protein.

Liposomes offer several advantages: They are non-toxic and biodegradable in composition; they display long circulation half-lives; and recognition molecules can be readily attached to their surface for targeting to tissues. Finally, cost effective manufacture of liposome-based pharmaceuticals, either in a liquid suspension or lyophilized product, has demonstrated the viability of this technology as an acceptable drug delivery system.

Other controlled release drug delivery systems, such as nanoparticles and hydrogels may be potential delivery vehicles for the subject vectors. These carriers have been developed for chemotherapeutic agents and protein-based pharmaceuticals, and consequently, can be adapted for nucleic acid delivery.

Chemical modification of the nucleic acid sequences encoding the subject vectors neutralizing negative charge may be all that is required for penetration. However, in the event that charge neutralization is insufficient, the subject vectors can be co-formulated with permeability enhancers, such as Azone or oleic acid, in a liposome. The liposomes can either represent a slow release presentation vehicle in which the subject vectors and permeability enhancer transfer from the liposome into the targeted cell, or the liposome phospholipids can participate directly the subject vectors and permeability enhancer can participate directly thereby facilitating cellular delivery. In some cases, both the subject vectors and permeability enhancer can be formulated into a suppository formulation for slow release.

The subject vectors may also be systemically administered. Systemic absorption refers to the accumulation of drugs in the blood stream followed by distribution throughout the entire body. Administration routes which lead to systemic absorption include: intravenous, transcutaneous, intramuscular, subcutaneous, intraperitoneal, intranasal, intrathecal and ophthalmic. A gene gun may also be utilized. Administration of DNA-coated microprojectiles by a gene gun requires instrumentation but is as simple as direct injection of DNA. A gene construct is precipitated onto the surface of microscopic metal beads. The microproj ectiles are accelerated with a shock wave or expanding helium gas, and penetrate tissues to a depth of several cell layers. This approach permits the delivery of foreign genes to the skin of anesthetized animals. This method of administration achieves expression of transgenes at high levels for several days and at detectable levels for several weeks. Each of these administration routes exposes the subject vectors to an accessible targeted tissue. Subcutaneous administration drains into a localized lymph node which proceeds through the lymphatic network into the circulation. The rate of entry into the circulation has been shown to be a function of molecular weight or size. The subject vectors can be modified to diffuse into the cell, or the liposome can directly participate in the delivery of either the unmodified or modified vectors to the cell. Liposomes injected intravenously show accumulation in the liver, lung and spleen. The composition and size can be adjusted so that this accumulation represents 30% to 40% of the injected dose. The remaining dose circulates in the blood stream for up to 24 hours.

Alternatively, another method of administration involves the use of a DNA transporter system for inserting specific DNA into a cell. The DNA transporter system comprises a plurality of a first DNA binding complex, the complex including a first binding molecule capable of non-covalently binding to DNA, the first binding molecule covalently linked to a surface ligand, the surface ligand capable of binding to a cell surface receptor; a plurality of a second DNA binding complex, the complex including a second binding molecule capable of non-covalently binding to DNA, the second binding molecule covalently linked to a nuclear ligand, the nuclear ligand capable of recognizing and transporting a transporter system through a nuclear membrane; wherein the plurality of first and second DNA binding complexes are capable of simultaneously, non-covalently binding to a specific DNA.

Additionally, a plurality of a third DNA binding complex may be used, the complex includes a third binding molecule capable of non-covalently binding to DNA, the third binding molecule covalently linked to a virus; wherein the plurality of third DNA binding complexes are capable of simultaneously, non-covalently binding to a specific DNA.

The first binding molecule, the second binding molecule and third binding molecule can each be selected from the group consisting of spermine, spermine derivative, histones, cationic peptides and polylysine. Spermine derivative refers to analogues and derivatives of spermine and include compounds as set forth in International Publication No. WO 93/18759, filed Mar. 19, 1993 and published Sep. 30, 1993, hereby incorporated by reference.

Establishment of therapeutic levels of the subject vectors are dependent upon the rates of uptake and degradation. Decreasing the degree of degradation will prolong the intracellular half-life of the subject vectors.

The subject vectors may be administered utilizing an ex vivo approach whereby cells are removed from an animal, transduced with the subject vectors and reimplanted into the animal. The liver can be accessed by an ex vivo approach by removing hepatocytes from an animal, transducing the hepatocytes in vitro with one or more subject vectors and reimplanting them into the animal (e.g., as described for rabbits by Chowdhury et al., Science 254:1802-1805, 1991, or in humans by Wilson, Hum. Gene Ther. 3:179-222, 1992) incorporated herein by reference.

The subject vectors may be administered utilizing an in vivo approach whereby the gene will be administered directly to an animal by intravenous injection, intramuscular injection, or by catheterization and direct delivery of the gene via the blood vessels supplying the target organ.

While the methods of administration discussed herein focus on the administration of viral vectors, those skilled in the art will appreciate that many of the methods can be routinely tailored to use and administration of naked DNA and/or proteins as vaccination systems.

### Production of viral vectors

Adenoviruses. Those skilled in the art will appreciate that for viral DNA replication and packaging of viral DNA into virion particles, only three regions of the viral DNA are known to be required in cis. These are the left inverted terminal repeat, or ITR, (bp 1 to approximately 103) the packaging signals (approximately 194 to 358 bp) (Hearing and Shenk, 1983, Cell 33: 695-703; Grable and Hearing 1992, J. Virol. 64: 2047-2056) and the right ITR. Among the regions of the viral genome that encode proteins that function in trans, two have been most important in the design and development of adenovirus vectors. These are early region 3 (E3) located between approximately 76 and 86 mu (mu=% distance from the left end of the conventionally oriented genome) and early region 1 (E1) located between approximately 1 and 11 mu. E3 sequences have long been known to be nonessential for virus replication in cultured cells and many viral vectors have deletions of E3 sequences so that the capacity of the resulting vector backbone for insertion of foreign DNA is thereby increased significantly over that allowable by the wild-type virus (Bett, A. J., Prevec, L., and Graham, F. L. Packaging capacity and stability of human adenovirus type 5 vectors. J. Virol. 67: 5911-5921, 1993.). E1 encodes essential functions. However, E1 can also be deleted, providing that the resulting virus is propagated in host cells, such as the 293 cell line, PER-C6 cells, 911 cells, and the like, which contain and express E1 genes and can complement the deficiency of E1(=) viruses.

Viruses with foreign DNA inserted in place of El sequences, and optionally also carrying deletions of E3 sequences are conventionally known as “first generation” adenovirus vectors. First generation vectors are of proven utility for many applications. They can be used as research tools for high-efficiency transfer and expression of foreign genes in mammalian cells derived from many tissues and from many species. First generation vectors can be used in development of recombinant viral vaccines when the vectors contain and express antigens derived from pathogenic organisms. The vectors can be used for gene therapy, because of their ability to efficiently transfer and express foreign genes in vivo, and due to their ability to transduce both replicating and nonreplicating cells in many different tissues. Adenovirus vectors are widely used in these applications.

There are many known ways to construct adenovirus vectors. As discussed above, one of the most commonly employed methods is the so called “two plasmid” technique. In that procedure, two noninfectious bacterial plasmids are constructed such that each plasmid alone is incapable of generating infectious virus. However, in combination, the plasmids potentially can generate infectious virus, provided the viral sequences contained therein are homologously recombined to constitute a complete infectious virus DNA. According to that method, typically one plasmid is large (approximately 30,000-35,000 nt) and contains most of the viral genome, save for some DNA segment (such as that comprising the packaging signal, or encoding an essential gene) whose deletion renders the plasmid incapable of producing infectious virus. The second plasmid is typically smaller (eg 5000-10,000 nt), as small size aids in the manipulation of the plasmid DNA by recombinant DNA techniques. Said second plasmid contains viral DNA sequences that partially overlap with sequences present in the larger plasmid. Together with the viral sequences of the larger plasmid, the sequences of the second plasmid can potentially constitute an infectious viral DNA. Cotransfection of a host cell with the two plasmids produces an infectious virus as a result of homologous recombination between the overlapping viral DNA sequences common to the two plasmids. One particular system in general use by those skilled in the art is based on a series of large plasmids known as pBHG10, pBHG11 and pBHGE3 described by Bett, A. J., Haddara, W., Prevec, L. and Graham, F. L: An efficient and flexible system for construction of adenovirus vectors with insertions or deletions in early regions 1 and 3,” Proc. Natl. Acad. Sci. US 91: 8802-8806, 1994 and in U.S. patent application Ser. No. 08/250,885, and published as WO95/00655 (hereby incorporated by reference). Those plasmids contain most of the viral genome and are capable of producing infectious virus but for the deletion of the packaging signal located at the left end of the wild-type viral genome. The second component of that system comprises a series of “shuttle” plasmids that contain the left approximately 340 nt of the Ad genome including the packaging signal, optionally a polycloning site, or optionally an expression cassette, followed by viral sequences from near the right end of E1 to approximately 15 mu or optionally to a point further rightward in the genome. The viral sequences rightward of El overlap with sequences in the pBHG plasmids and, via homologous recombination in cotransfected host cells, produce infectious virus. The resulting viruses contain the packaging signal derived from the shuttle plasmid, as well as any sequences, such as a foreign DNA inserted into the polycloning site or expression cassette located in the shuttle plasmid between the packaging signal and the overlap sequences. Because neither plasmid alone has the capability to produce replicating virus, infectious viral vector progeny can only arise as a result of recombination within the cotransfected host cell. Site-specific methods for achieving recombination may also be employed when practicing the present invention.

It has been shown that use of hdAds can lead to prolonged transgene expression and reduced immune and inflammatory responses compared to first generation Ad vectors. HdAds retain the other beneficial properties of Ad vectors, mainly virion stability during vector propagation and purification, and high transduction efficiency of replicating and quiescent cells, while eliminating some of the obstacles and concerns that have been raised with respect to first-and second-generation Ads. Should transgene expression levels decrease over time, the use of hdAds of alternative serotypes may permit readministration of a vector with the identical genotype. Since vector persistence (and hence transgene expression) is influenced by immune responses to both vector and transgene, the effectiveness of vector readministration using hdAd's may ultimately depend primarily on the immunogenicity of the therapeutic gene. Accordingly, in the absence of transgene effects, the sequential use of hdAd of alternative serotype can be an effective strategy for vector readministration. Accordingly, therapeutic genes encoding products of low immunogenicity may be repeatedly administered according to the instant disclosure. In addition, in vaccine applications, in which repeat administration of a gene encoding a particular gene product against which an immune response is desired, or when administration of a second, third, fourth etc. gene is desired, ability to overcome unwanted immune responses induced by a previous exposure to a vector is highly desirable.

Other viral vectors. Other various viral vectors can be utilized to practice the subject invention, including, but not limited to, adeno-associated virus, herpes virus, vaccinia, or an RNA virus, such as an alpha virus. Preferably, the retroviral vector is a derivative of a murine or avian retrovirus. Preferably, the alphavirus vector is derived from Sindbis or Semliki Forest Virus. Examples of retroviral vectors in which a single foreign gene can be inserted include, but are not limited to, Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. An alphavirus vector for use in the method of this invention comprises a recombinant alphavirus vector system which expresses the lac Z gene. Construction of this vector is described in P. Liljestrom, Current Opin. Biotechnol5(5):495-500, 1994; and P. Liljestrom et al., Biotechnology (NY) 9(12):1356-61, 1991.

The teachings of all of the references cited throughout this specification are incorporated by reference to the extent they are not inconsistent with the teachings herein.

### EXAMPLES

### Example 1: Construction of Recombinant Plasmids and Adenovirus Containing a Functional Coding Gene for Propionibacterium Acnes Lipase

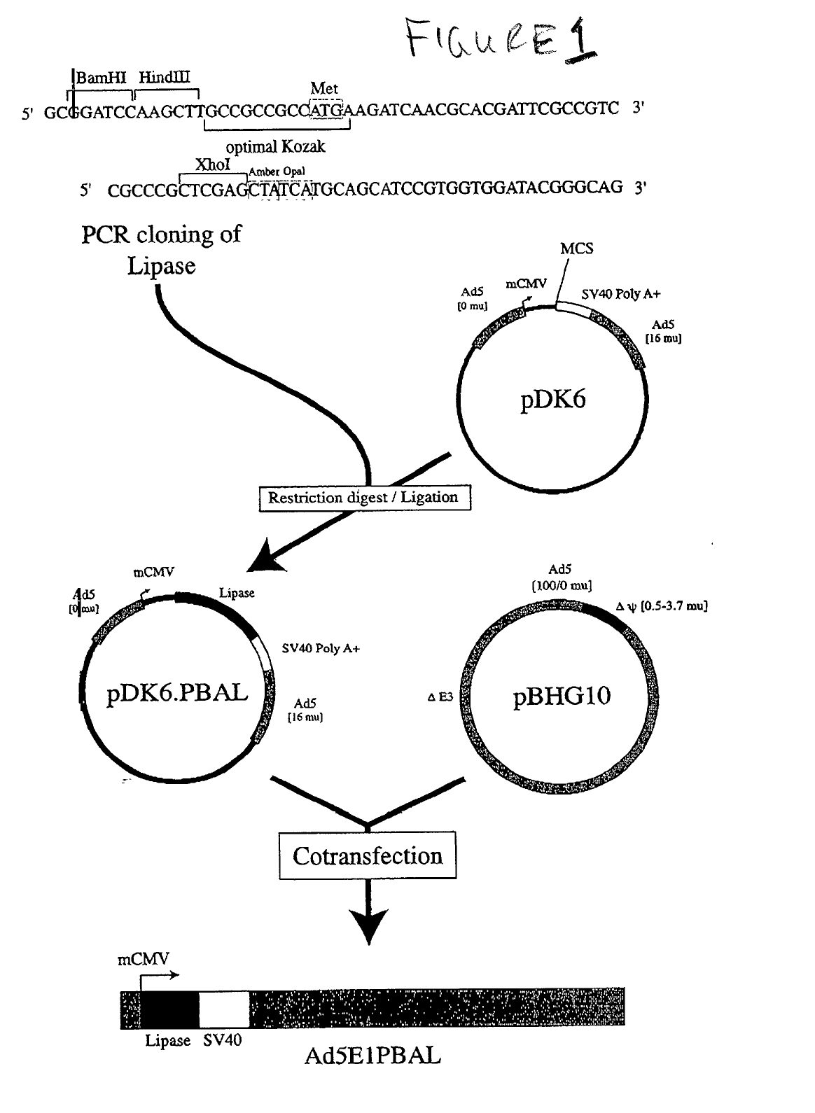

A schematic diagram for the construction of the Ad5E1PBAL vector is shown in FIG. 1. To rescue the Propionibacterium acnes lipase sequences into a translatable minigene cassette, an oligonucleotide was designed containing 5′ flanking restriction enzyme sites for Bam HI and Hind III, followed subsequently by a sequence coding for the consensus optimal ribosomal translation initiation site, and bases incorporating the first 30 nucleotides of the coding sequence for P. acnes lipase gene. The following is the sequence of the 5′ oligonucleotide: GCGGATTCCAAGCTTGCCGCCG-CCATGAAGATCAACGCACGATTCGCCGTC. An additional oligonucleotide containing bases complementary to the 3′ end of the P. acnes lipase gene flanked by residues containing stop codons to provide a translational termination signal and a restriction site Xho I was created. The sequence of the 3′ oligonucleotide is: CGCCCGCTCGAGCTA-TCATGCAGCATCCGTGGTGGATACGGGCAG. Additional nucleotides were incorporated in the design of the 5′ and 3′ oligonucleotides to accommodate for restriction enzyme cleavage activity at blunt ends of DNA. PCR reactions were carried out using the 5′ and 3′ designed oligonucleotides with genomic DNA isolated from P. acnes bacteria.

An approximate 1 kb PCR fragment was isolated and subcloned by blunt end ligation into pCR-Blunt. This fragment was flanked by Bam HI and Hind III at the 5′ end with an optimal consensus Kozak with 3′ stop codon sequences flanked by an Xho I restriction site. The Bam HI, Hind III and Xho I restriction sites were included so that the P. acnes lipase gene could be fragmented to generate variant determinant targets since the Bam HI and Hind III sites are present within the gene. This will allow the rescue of mini-genes encoding portions of the lipase gene to be cloned into the polylinker site of pDK6. The lipase sequence was rescued from the blunt vector by Kpn I and Xho I digest and cloned into these sites in the pDK6 vector. This construction places the transgene under the control of the murine cytomegalovirus (mCMV) promoter and provides polyadneylation signals from the simian virus 40 (SV40). To obtain the resultant adenovirus vector expressing the P. acnes lipase gene, pDK6PBAL DNA was cotransfected with pBHG10 into 293 cells using standard adenovirus rescue protocols. One viral plaque was identified by restriction enzyme digest, Southern blot and by sequence to contain the P. acnes lipase gene sequence and was designated as Ad5E1PBAL vector. This recombinant vector was propagated in 293 cells and purified by cesium chloride gradient centrifugation and dialysis before use in the animal studies. All oligonucleotides used in this study were obtained from Integrated DNA Technologies, Inc., Coralville, IA.

### Example 2: Successful Treatment of Acne Vulgaris.

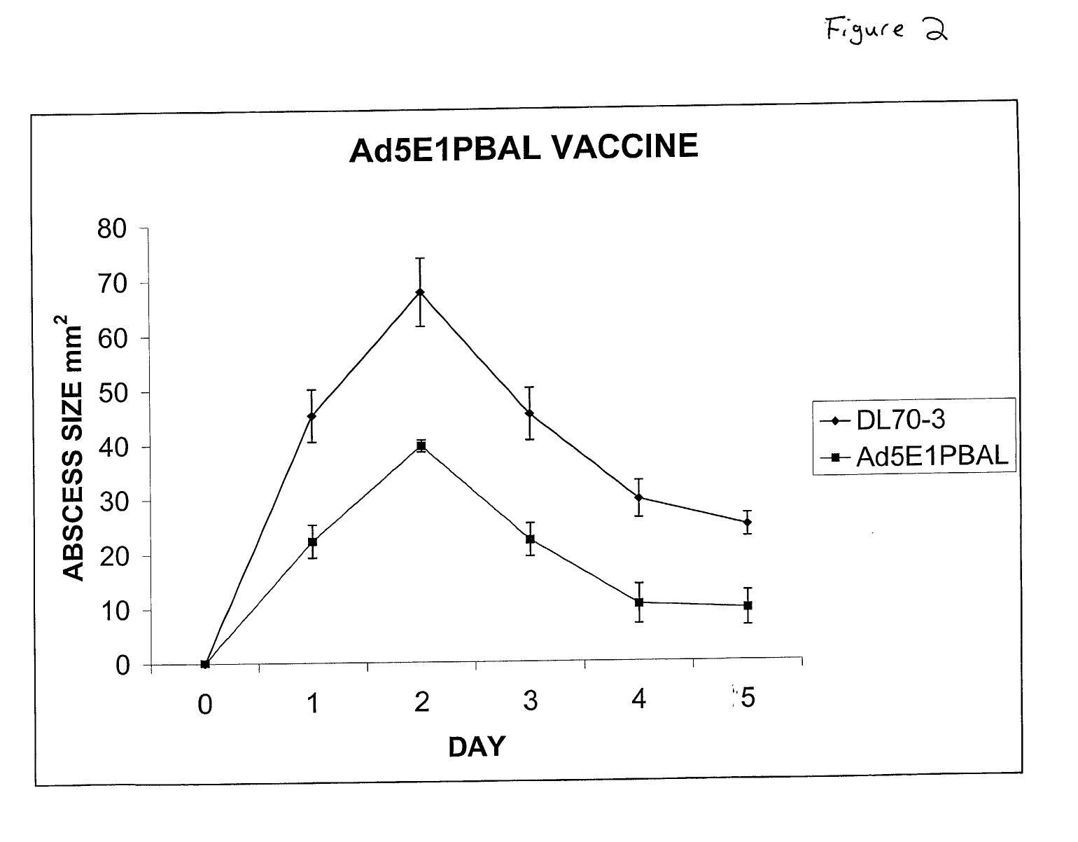

Balb/c mice were purchased from the Taconic labs and bred under specific pathogen-free conditions in the McMaster University animal facility. Female mice were used at 8-14 wk of age. Mice were immunized intramuscularly with 2×10⁹ pfu in 50μl saline of AdEl Lipase (Ad5ElPBAL) or control (empty) vector (DL70-3) I.M on left hind leg. 7 days later disease was induced by injection of 100μl of 1×10⁹ cfu/ml of P. acnes intramuscularly in PBS on left rear flank. All recombinant viruses were propogated and purified as described for the Ad5ElPBAL vector. Control vector DL70-3 is an Ad5 variant deleted in the E1 region. All reactions were measured by caliper sizing. FIG. 2 demonstrates that pre-immunization with lipase of P. acnes provided protections from P. acnes challenge. All work was performed in accordance with McMaster University guidelines for animal use and care. The foregoing examples are for illustration purposes only and should not be construed as limiting the scope of the subject invention.

What is claimed is:

1. A vaccine useful in preventing and treating diseases caused by a pathogen capable of infecting, or avoiding destruction by, macrophages, said vaccine comprising at least one vector that comprises at least one nucleotide sequence encoding at least one antigen derived from said pathogen, and wherein said antigen is capable of generating an immune response in a recipient thereof.

2. The vaccine of claim 1, wherein said pathogen is P. acnes, L. monocytogenes, S. typhimurium, N. gonorrhoea, M. avium, M. tuberculosis, M. leprae, B. abortus, C. albicans; L. major, or combinations thereof.

3. The vaccine of claim 2, wherein said pathogen is P. acnes.

4. The vaccine of claim 1, wherein said vector comprises naked DNA, a recombinant viral vector, or a combination of both.

5. The vaccine of claim 4, wherein said recombinant viral vector is selected from the group consisting of adenovirus, adeno-associated virus, herpes virus, vaccinia and RNA viruses.

6. The vaccine of claim 5, wherein said recombinant viral vector is an adenovirus.

7. The vaccine of claim 1, wherein said vector further comprises a nucleotide sequence encoding an adjuvant.

8. The vaccine of claim 7, wherein said adjuvant is a cytokine.

9. The vaccine of claim 8, wherein said cytokine is IL-2, IL-12, or both.

10. The vaccine of claim 1, wherein said antigen is a lipase gene or fragments thereof, a hyaluronidase gene or fragments thereof, a phosphatase gene or fragments thereof, or combinations of the foregoing.

11. A method of treating or preventing a disease caused by a pathogen capable of infecting, or avoiding destruction by, macrophages, said method comprising obtaining a vaccine comprising at least one vector that comprises at least one nucleotide sequence encoding at least one antigen derived from said pathogen; and administering said vaccine to a recipient in need thereof.

12. The method of claim 11, wherein said adminstering comprises routes of adminstration comprising oral, intravenous, intramuscular, transcutaneous, subcutaneous, aerosol, ocular, rectal, intraperitoneal, intrathecal, or combinations thereof.

13. The method of claim 12, wherein administering comprises transcutaneous administration.

14. The method of claim 13, wherein said transcutaneous administration comprises applying said at least one vector to a patch, and adhering said patch to skin of said recipient.

15. The method of claim 1 1, wherein said pathogen is P. acnes, L. monocytogenes, S. typhimurium, N. gonorrhoea, M. avium, M. tuberculosis, M. leprae, B. abortus, C. albicans; L. major, or combinations thereof.

16. The method of claim 15, wherein said pathogen is P. acnes.

17. The method of claim 11, wherein said at least one vector comprises naked DNA, a recombinant viral vector, or a combination of both.

18. The method of claim 17, wherein said recombinant viral vector is an adenovirus.

19. A kit comprising a container and one or more patches, wherein said patches have disposed thereon at least one vector comprising a nucleotide sequence encoding an antigen derived from a pathogen, said pathogen being capable of infecting, or avoiding destruction by, macrophages.

20. An article of manufacture comprising a vaccine solution disposed within a tube, vial, bottle, can, or syringe, wherein said vaccine solution comprises a viral vector comprising a nucleotide sequence encoding an antigen derived from a pathogen, said pathogen being capable of infecting, or avoiding destruction by, macrophages.

21. The vaccine of claim 1, wherein said vaccine is in the form of an aqueous solution.

22. The vaccine of claim 1, wherein said vaccine further comprises a nucleotide sequence encoding a co-stimulatory molecule.

23. The vaccine of claim 22, wherein said co-stimulatory molecule comprises a B7 protein, a CD40 protein or both.

24. A method of cosmetically improving the appearance of a person's skin who is suffering from acnes vulgaris, said method comprising the steps of obtaining a composition comprising a mixture of at least one vector that comprises at least one nucleotide sequence encoding at least one antigen derived from said P. acnes, and a cosmetic agent; and administering said composition to said person.
